(19) United States
(12) Patent Application Publication
(10) Pub. No.: US 2026/0137374 A1
(43) Pub. Date: May 21, 2026

(54) REMOTE DIAGNOSTICS AND TREATMENT SYSTEMS

(71) Applicant: INNOSCION LLC, San Francisco, CA (US)

(72) Inventor: Theodore P. Abraham, San Francisco, CA (US)

(21) Appl. No.: 18/705,866

(22) PCT Filed: Nov. 1, 2022
(86) PCT No.: PCT/US2022/048548

§ 371 Date: Apr. 29, 2024

Related U.S. Application Data
(60) Provisional application No. 63/274,396, filed on Nov. 1, 2021.

(51) Int. Cl. A61B 8/00; A61B 8/08; A61B 10/02; A61N 7/00; G16H 40/67

(52) U.S. Cl. CPC A61B 8/565; A61B 8/0866; A61B 8/0883; A61B 8/4236; A61B 8/4494; A61B 8/54; A61B 10/02; A61N 7/00; G16H 40/67

(57) ABSTRACT

Systems and methods for remote diagnostics and therapies are disclosed. A remote technician or automated computer system, optionally using artificial intelligence algorithms, can communicate with and control home diagnostic and therapeutic devices such as ultrasound imaging devices avoiding the need for patient travel and costly hospital visits. Devices can be delivered via standard courier services or other mechanisms such as drones or self-driving delivery vehicles. Once delivered a remote diagnostic and/or therapy session can be completed using a wired or wireless communications network. Portions of the devices can then be disposed of or returned for redistribution. Devices may include security measures such as user lockouts after authorized use is complete and/or self-destruction mechanisms to prevent tampering or unauthorized use of expensive or proprietary components.

### CROSS-REFERENCE TO RELATED APPLICATIONS

This Application claims the benefit of, and priority to, U.S. Provisional Application No. 63/274,396, filed Nov. 1, 2021, the content of which is hereby incorporated by reference in its entirety.

### FIELD OF THE INVENTION

Systems and methods of the invention relates generally to remote diagnostic and treatment systems including remote ultrasound and other imaging and testing modalities.

### BACKGROUND

The Federal Communications Commission (FCC) has estimated that the health care industry could save $700 billion in the next 15 to 20 years with the use of remote patient monitoring technology in conjunction with electronic health records (EHR). However, remote monitoring technologies are still lacking and more sophisticated technologies often require trained medical professionals, sometimes in person, to run. One diagnostic technology that lends itself to remote use, however, is ultrasound imaging. As discussed in U.S. Pat. No. 8,038,622, incorporated herein by reference, remote ultrasound devices can be deployed by patients and monitored remotely, providing relatively low cost, portable, radiation-free imaging (compared to X-ray, CT, and MRI techniques).

Ultrasound imaging is known in the art and systems typically involve the use of hand-held or temporarily affixed ultrasound transducer arrays that may be controlled, for example, as to on/off, mode, focus control, depth control and the like. The user applies a small amount of ultrasound gel to a region of interest, holds and moves the ultrasound transducer from one location of the patient to another within the region, the unit being wired to a console, typically including a display. Ultrasound is a biologically safe and non-radiating form of energy that can provide detailed anatomic and, in some cases, functional images. It is known in the art of transesophageal echocardiography (imagery of the heart) to provide a multi-plane transducer that can image in planes in a 180 degree range.

It is also known, for example, in the telecommunications arts to remotely transmit images such as photographic images from a source such as a cellular telephone device to a receiving cellular telephone or other telecommunications device. For example, a doctor may transmit a digital image to another doctor by attaching the image to an email. An x-ray machine located in a remote laboratory may capture an image of a broken bone, and the technician may immediately transmit the image to an orthopedic unit of a hospital for analysis. Cellular telephone devices are now capable of capturing and transmitting moving images, including movies with associated sound, for personal enjoyment.

### SUMMARY

Systems and methods of the invention relate to remote diagnostic and treatment systems that allow a patient to perform a variety of diagnostic and/or treatment procedures with limited or no on-site assistance. Through the use of portable devices and remote control and/or instruction provided by automated computer programs, artificial intelligence, remote technicians, or a combination thereof, healthcare costs can be reduced along with patient inconvenience (and resulting non-compliance) associated with travelling to healthcare providers. Patients may put off important diagnostic tests or treatments due to inconvenience or actual logistical difficulties with traveling to a hospital or other medical facility. When healthcare is delayed, early diagnosis and treatment opportunities can be missed resulting in significant additional costs and worse patient outcomes. By empowering patients to run their own tests and treatments, these issues can be avoided. Additionally, allowing for remote testing and therapy can help prevent the spread of disease by limiting the concentration of sick individuals in a hospital or other medical facility, an important consideration during outbreaks and events such as the present COVID-19 pandemic. Furthermore, by using automated programs including artificial intelligence (AI) augmented support, a patient is freed not only of geographic constraints on testing and treatment, but also time constraints as the patient can administer tests and treatment whenever is convenient. Additionally, by removing expensive trained medical professionals from many of the diagnostic and treatment modalities, costs are further reduced.

Remote diagnostic systems can include imaging devices such as the remote ultrasound devices described in U.S. Pat. No. 8,038,622 as well as other sensors such as simple cameras (e.g., otoscopes) as well as blood pressure, pulse oximetry, EKG, and temperature sensors. Devices can be delivered using existing courier services or through automated delivery via driverless vehicles or drones. Device hookup and testing/treatment can be performed by the patient or other individuals without the need for medical training or particular expertise.

Aspects of the invention can include methods and systems for performing remote medical procedures including: providing a medical device comprising a sensor in communication with a transmitter comprising a processor and a tangible, non-transient memory connected to a network, the medical device operable to obtain diagnostic tests on a patient; initiating communication between the transmitter and a remote workstation; receiving patient information from the sensor at the remote workstation; providing commands to the medical device from the remote workstation to control the medical device to perform a medical procedure.

In certain embodiments, methods may include instructing the patient to setup the medical device via the transmitter. The transmitter may be a patient computer and methods can further comprise providing software to the patient computer to communicate with the remote workstation and medical device. In various embodiments, methods may include delivering the medical device to the patient. The medical device can be delivered by a drone or a self-driving vehicle. The medical device may comprise a security feature. The security feature may include a lockout preventing subsequent use of the medical device after completion of the medical procedure. The security feature can comprise a self-destruct mechanism rendering the medical device inoperable upon unauthorized disassembly or use of the medical device. The security feature may include an electronic tracking mechanism.

In certain methods of the invention, the sensor may be selected from the group consisting of an ultrasound device, an impedance sensor, a plethysmography device, an optical coherence device, an infrared sensor, an acoustic sensor, an electrocardiograph device, an electromyography device, an electroencephalography device, a pulse sensor, a blood pressure sensor, an oxygen saturation sensor, an elastography device, a Raman spectrometer, and a tissue collecting device. Where the sensor comprises a tissue collecting device, the medical device may include a sterile and temperature-controlled tissue storage container, the medical device operable to store a collected tissue sample from the tissue collecting device in the storage container.

Medical devices of the invention may be operable to deliver a therapy to the patient and methods may include providing commands to the medical device from the remote workstation to deliver the therapy to the patient based on the patient information received from the sensor. In various embodiments, the therapy can include delivery of thermal energy to the patient; oral, transdermal, intravenous, or inhalation delivery of a therapeutic compound to the patient; iontophoresis-aided drug delivery; radiation therapy; chemotherapy; clotting therapy; anti-clotting therapy; laser ablation; phototherapy; acupuncture; electrical stimulation; magnetic stimulation; topical therapy; oral therapy; dental therapy; eye treatment; musculoskeletal therapy; abdominal therapy; or pelvic therapy.

In certain embodiments, one or more of the receiving patient information and providing commands steps can be performed by a technician at the remote workstation. One or more of the receiving patient information and providing commands steps can also be performed automatically by a computer comprising a non-transitory memory and a processor. The computer may provide the commands based on an analysis of the patient information received using one or more artificial intelligence (AI) algorithms. AI or machine learning technologies can be used for a variety of functions of the invention according to various embodiments. For example, operation of the imaging, diagnostic, and therapy delivery functions as discussed above, but also rapid disease diagnosis based on the information obtained by the remote diagnostic devices and sensors. AI and/or machine learning technologies can also be used to assist a remote operator instead of providing direct control (e.g., by suggesting areas to image or additional diagnostic tests to be performed). AI and/or machine learning technologies and software engines can also be applied to test ordering, appointment scheduling (e.g., to maximize efficiency or pair patients with remote operators based on time, test proficiency, costs, language, or compatibility), test and/or therapy triage when device availability is limited, and device transportation.

Embodiments and aspects described herein relate generally to embodiments and aspects and methods of use of a wired or wireless transducer array that may be remotely controlled to capture multiple image planes at a region of interest of a patient or victim and manipulated or moved to different locations on the skin surface without having to have an operator or large ultrasound system apparatus present at the site of the patient or victim (hereinafter, simply, patient).

In accordance with one embodiment, a transducer array unit for fixing to a patient's body is wireless (or wired) and communicates imaging data collected by a typically linear ultrasound array by wireless (or wired) means to an external site where collected imaging data is displayed and may be viewed by an operator. One embodiment of a wireless transducer unit is very much like a probe that can be affixed to the body surface and its imaging functions controlled remotely, for example, by wireless radio telecommunication such as WiFi, Wimax, blue tooth or other radio frequency communication protocol. The wireless communication may also be ultrasound, infrared or utilize other wireless communication frequencies. The wireless or wired link may be a local or long-distance telecommunications link involving satellite transmission. The transducer may be any ultrasound transducer (mechanical, annular, phased array or linear array) and may be single or multi-dimensional. Each transducer array unit may have its own unique identification code which is communicated with each wireless or telecommunications transmission to a host site of ultrasound imaging processing, control and display. The unique code of the remotely manipulatable transducer array unit is used by a host ultrasound imaging remote site to communicate with it. The transducer array unit is, for example, battery powered and self-contained such that it may be worn by a patient with minimal invasion of the patient's privacy. It includes in primary part, ultrasound transducer circuitry for transmitting and receiving ultrasound waves, control leads for mode, depth, focus and the like as is known in the art but additionally includes control leads for controlling the movement of the transducer array and the direction of its transmission as well as image data transmission and control data reception circuitry. The transducer assembly thus contains a transducer array or element unit that is remotely manipulatable.

The patient may eat, sleep or, otherwise, function, for example, in or out of their hospital bed while the unit remotely views and transmits imaging data to the remote site. The unit of this embodiment fixed to the patient's body may comprise a linear array of transducers or a single transducer element that may be remotely controlled to rotate from one position to another, either clock-wise or counter-clock-wise to obtain a different planar view of the body part under analysis. The shape of the transducer unit may be round for multi-plane or square or rectangular for other transducers. The wireless transducer unit may function with a three dimensional imaging system allowing stereotactic and remote/robotic operation of devices delivered through or in conjunction with the unit as will be further described below. The transducer array or element may be fixed to a rotor and the rotor assembly and transceiver circuitry housed within a housing having for example a cylindrical shape with one side intended to be facing the patient's body. The flat side facing the body may have a layer of body impedance matching material complimentary to any gel application. A micromotor and optional associated gear assembly may incrementally rotate the transducer array or element, for example, through a range of 180 degrees. Its position may be remotely determined and stored in local or remote memory and/or displayed at the external remote control site. The size of the footprint of a housing for a remotely manipulatable transducer unit on a patient's body surface may be as small as 1 cm or as large as several centimeters in diameter (or length/width). Typical operating frequency of the ultrasound transducer array or element may be between 200 kHz and 100 MHz depending on the clinical application. In addition to rotation, a linear transducer array may be adaptably mounted to a rotor shaft so that it may also redirect output sound waves within a range of 180 degrees within the patient's body at the given angle of rotation. A first transducer array or element may cooperate with a second transducer array unit situated inches away as a transmitter while the second device operates as a receiver and vice versa. The first and second transducer array units may separately provide image data of the same region of interest to a remote workstation. The rotatable transducer embodiment may, for example, of circular or cylindrical shape and may be affixed to the body by a broad securing material that may be either adhesive or non-adhesive, such as a band or bandage of cloth or other fiber. No operator need be attendant at the patient site. Imaging data may be converted from analog to digital format and compressed before it is transmitted in accordance with well known standards to conserve transmission bandwidth. Typical ultrasound imaging bandwidth requirements should be on the order of 1 MHz. If high levels of resolution are required, the bandwidth may exceed 5 MHz or, if low resolution is permitted, a 100 kHz bandwidth may suffice.

In yet another embodiment, the patient may be an out-patient and wear a battery-powered transducer array or transducer element apparatus that may be remotely monitored and manipulated via a telecommunications channel or by the out-patient via a cord to a remote control. Image data may be collected and stored locally in removable or accessible memory over time. The out-patient may visit their doctor and the memory contents unloaded rather than be remotely transmitted. On the other hand, the memory, when it reaches a predetermined fill capacity may transmit its contents via the transceiver to a remote workstation for analysis. If a remote workstation operator sees an extraordinary condition in ultrasound imagery, the out-patient may be warned by the operator's triggering a vibration or other alarm of such condition via a telecommunications or wireless link that the out-patient must see their doctor immediately. The operator may continuously monitor, for example, from the remote site for signs of patient difficulty. For example, the development of a blood clot or other serious condition may be viewed remotely if the device is used in conjunction with, for example, a knee replacement operation.

The out-patient may control the transducer array or element themselves, for example, to deliver therapeutic ultrasound waves to a region of interest to them and so manipulate the transducer to change a direction of propagation of sound waves from one set for imaging by a remote operator.

In another alternative embodiment, the transducer array unit may be formed as a square or a rectangle and the linear transducer array in addition to rotation, twist or direction of sound wave transmission may move under remote motor control in a lengthwise direction from one end of a square or rectangular shaped housing to the other. In another embodiment as described above, the array may move in two perpendicular directions, for example, in an x or y axis direction on the body surface and not be permitted to rotate. Such a device may comprise a single transducer element or a linear array. On the other hand, a transducer array, for example, contained in a square or rectangular housing may also be rotatable to a predetermined angle of rotation by remote control at each incremental lengthwise or widthwise position and/or twisted. For example, such a transducer array unit may be used to monitor a fetus within a patient as it moves within the abdominal cavity. One unit may be fixed to a female patient's body and be manipulated alone or in conjunction with another or plural remotely manipulatable transducer units affixed to the female patient's body.

In an alternative embodiment and in conjunction with an imaging catheter as described in my co-pending U.S. patent application Ser. No. 11/782,991, filed Jul. 25, 2007, one or more remotely manipulatable transducer units may be used together with the imaging catheter to provide additional imaging of a minimally invasive heart operation or other procedure being performed on a patient in an operating arena. In deed visualization of any body part is possible including the heart, liver, kidney, brain, prostrate, any vascular structure, gland (such as the thyroid), extremity (knee replacement) or other body part to be monitored. For example, the remotely manipulatable wireless transducer unit may facilitate any intervention requiring ultrasound guidance including but not limited to entry into various body spaces such as pleural, peritoneal and pericardial space thus allowing therapy delivery, intervention, placement of devices such as pacemakers or medicine pumps and diagnostics. In addition to an imaging catheter, one or more remotely manipulatable wireless transducer array units may be used with another interventional, therapeutic or diagnostic system such as a biopsy forceps, a drainage catheter, a pressure monitoring system, a suture application system, a therapy delivery system or other interventional, therapeutic or diagnostic system known in the art.

In yet another embodiment, the remotely manipulatable transducer array unit may deliver ultrasound energy for therapeutic rather than imaging purposes, for example, to specific locations on or under the skin surface or within the body. During an interventional or a therapeutic procedure, the interventional procedure requiring intermittent ultrasound monitoring such as surgery or cardiac catheterization, the transducer can remain on the body during the entire procedure and the imaging or therapeutic treatment performed as and when needed by the remote operator. The remote operator may communicate with a surgeon or other operating room personnel by telecommunications to, for example, report that a medicine pump has been properly placed and is operating, for example, via a headset worn by the surgeon or other operating room personnel within an operating room.

The remotely manipulatable wireless transducer array unit communicates with a remote workstation that may include at least one display and a user interface screen for not only viewing an imaged area but, for example, a compartmentalized image area of a plurality of image displays of a region of interest and additionally present a user interface providing, for example, time of day, rotational degree and other control feedback in conjunction with usage of a control device such as a trackball or mouse. Other known controls such as on/off, depth, gain, focus and the like make be provided via conventional buttons, knobs or monitor screen controls. The workstation may comprise one or a plurality of displays of the transmitted image of a region of interest including a three dimensional display or plural displays of multiple planes or a display showing manipulation of the ultrasound transducer element within the boundaries of a housing as placed on a patient and/or a display of operating parameters such as the coordinates of location of the transducer, its angle of rotation and its twist or angle of sound transmission. If plural transducer units are utilized, the single workstation may provide additional displays for each remotely controlled and manipulatable transducer unit.

A first remote workstation may communicate with a second remote workstation by wired or wireless means and the second remote workstation may serve as a back-up to the first remote workstation in another embodiment. An operating room can only efficiently contain so many people assisting a surgeon and so much equipment. For example, the primary area of use of a wireless remote transducer unit may be within a fluoroscopy suite or an operating room such that one or more remotely manipulatable wireless remote transducer units communicates with a primary workstation and/or a secondary remote workstation outside the suite or room. In such a situation, it is convenient if each of the primary and secondary workstations are uniquely identified as are the wireless transducer units and only one workstation is able to remotely control one wireless remote transducer unit at a time while the other may be afforded monitoring privileges. As suggested above, a control operator of a workstation may communicate with the surgeon by means of a headset to answer questions a surgeon or other operating room personnel may have as a yes or no or advise of a successful procedure.

### BRIEF DESCRIPTION OF THE DRAWINGS

FIG. 1 comprises a top view and side view of a first plurality of embodiments and aspects of a multi-plane transducer unit comprising a rotatable linear array of transducer elements including a housing for mounting by securing material to a body of, for example, a patient or victim, which may be controllably rotated and otherwise controlled by wired or wireless signals remotely from the patient without an operator needing to be proximate to the body to manipulate or control the transducer elements or the housing.

FIG. 2 provides a schematic block diagram for embodiments and aspects of a device as shown in FIG. 1 which may be wireless and further including a transceiver in addition to a transducer control unit. Also shown are a battery power supply, at least one motor for rotating a linear transducer element array, the linear transducer element array and analog to digital circuitry for converting collected image data to digital form for transmission via the transceiver.

FIG. 3 provides an overview of a mechanical arrangement to be contained within a housing of embodiments of a transducer assembly unit for manipulating a transducer or linear transducer array in two perpendicular directions, for example, along an x axis and a y axis, to provide an angle of rotation to permit multiple image planes and a twist angle to redirect a sound wave emitted by a transducer or linear array of transducer elements whereby it is envisioned that a footprint on a patient body surface is rectangular or square and relates to the embodiments and circuits of FIGS. 1 and 2.

FIG. 4a provides an exemplary signal content format for providing motor control of a remotely manipulatable transducer or transducer array of FIG. 1, 2 or 3 in a direction from a workstation to a remote wired or wireless transducer including a unique transducer transceiver identifier if wireless or telecommunications transmission is utilized. The format also provides for known control such as on/off, focus, depth, mode and the like.

FIG. 4b provides an exemplary signal content format for providing a reply signal from a transducer or transducer array of FIG. 1, 2 or 3 in a direction from a remote wired or wireless transducer including a unique workstation identifier if wireless or telecommunications transmission is utilized. The depicted format provides for feedback of actual location data of the position of the transducer or transducer array.

FIG. 5 provides a plurality of suggested locations on a human body for locating a remotely manipulatable transducer or transducer array whereby the units may comprise devices and adhesive material similar to electrodes used in electrocardiography, banded adhesive attachment or bandage or banding of the manipulatable transducer or transducer array housing to a human patient's body.

FIG. 6 provides an exemplary application of a manipulatable transducer array that may be worn by a pregnant female in the abdominal area for remotely monitoring a fetus for, for example, extraordinary conditions.

FIG. 7 shows an arrangement for pericardial activity and possibly surgery including first and second manipulatable transducer units mounted to either side of, for example, an image guided catheter surgical location as one example of an application for minimally invasive heart surgery.

FIG. 8 shows a workflow scenario whereby the primary workstation is remote from a fluoroscopy suite, operating room and the like and, if necessary, shielded from adverse impacts of radiation such as magnetic resonance.

FIG. 9 shows another workflow scenario for describing, for example, remote field emergency use, operating room, fluoroscopy suite use and primary and alternative workstations connected by a wireless or wired telecommunications network.

FIG. 10 shows an exemplary workflow for certain methods of the invention.

FIG. 11 shows a detailed exemplary workflow for various methods of the invention according to certain embodiments.

### DETAILED DESCRIPTION

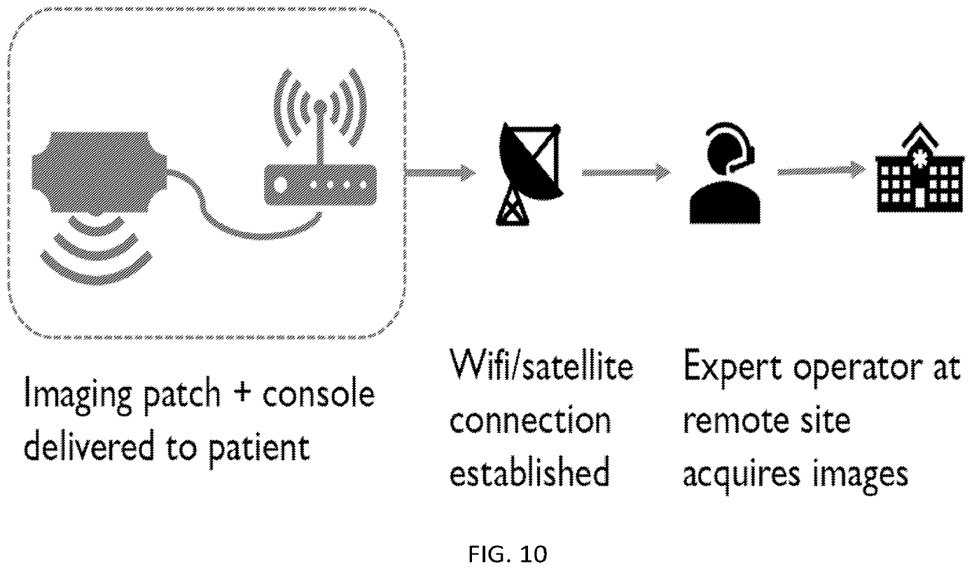

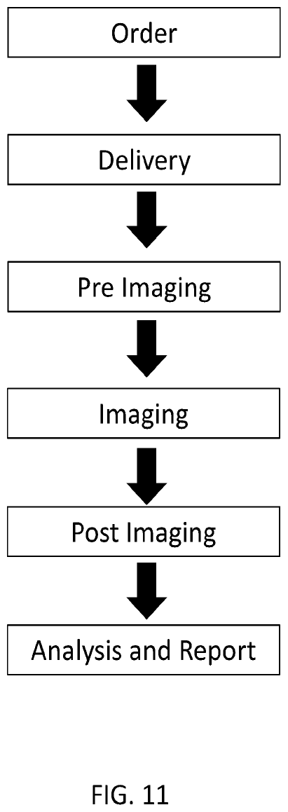

Exemplary workflows for various methods of the invention are shown in FIGS. 10 and 11. A simplified workflow is shown in FIG. 10 where an imaging patch or other diagnostic device as well as a console or transmitting device is delivered to a patient. As discussed below, in certain embodiments, the diagnostic device may rely on a patient's existing hardware (e.g., a laptop, tablet, or mobile phone) for communications and control. A remote connection is then established with a remote human operator, server housing automated instructions or an AI algorithm, or other remote device. The remote operator or automated device can then provide instructions to the patient and/or control the diagnostic device remotely to capture images or other diagnostic information.

FIG. 11 shows another exemplary workflow with additional detail. First a patient or care provider orders a particular diagnostic test (e.g., ultrasound imaging) after it is determined that bedside or home diagnostics are recommended.

The package including various combinations of imaging and diagnostic equipment and hardware/software for controlling the diagnostic equipment and enabling remote control and communication thereof are dispatched to the patient. The package may be delivered electronically in the case of software and any physical devices may be delivered via traditional post or through alternative services such as drone, emergency medical services, third-party ride sharing/delivery platforms or apps, or self-driving/automated delivery vehicles.

The remote appointment can be scheduled for any time after delivery of the device(s). The patient can then be registered and the device(s) can be tested. Pre-imaging information and/or guidance can be provided to the patient via live or automated text, images, or video. These instructions can include device set-up and connection information.

At the appointment time, imaging or other diagnostic procedures can be performed remotely by a remote technician and/or an automated program or AI-enabled program. Some diagnostic procedures may be fully remotely operated while some may require or benefit from patient or other local user manipulation. For example, certain procedures may require moving and orienting an imaging or other diagnostic device one or more times to provide more complete diagnostic information. The patient may be guided through device placement and/or other device setup actions through images, audio, video, or other cues provided via the associated delivered hardware or software. The guidance may be automated or may be provided in real-time by a remote operator or via AI algorithms and programming based on inputs provided by the delivered hardware/software. For example, diagnostic devices may use video cameras or other sensors to provide information to the remote operator for device positioning, etc. In the case of AI guidance/diagnostic testing, the sensors can provide input information for the AI algorithms.

Once the images or other diagnostic information is acquired, the remote technician or automated computer (optionally aided by AI analysis of the diagnostic information) may take one or more actions including generating a report, sending a report, conducting further diagnostic tests, or providing therapy. A report may be written and mailed or may be provided electronically to the patient, a treating physician or other medical professional, and insurance provider, or other interested party. Reports, along with any communication by, from, and between the remote workstation and the on-site medical device and transmitter may be encrypted or otherwise secured to protect sensitive patient data and as required by applicable laws and regulations. Therapies to be provided may include delivery of thermal energy to the patient; oral, transdermal, intravenous, or inhalation delivery of a therapeutic compound to the patient; iontophoresis-aided drug delivery; radiation therapy; chemotherapy; clotting therapy; anti-clotting therapy; laser ablation; phototherapy; acupuncture; electrical stimulation; magnetic stimulation; topical therapy; oral therapy; dental therapy; eye treatment; musculoskeletal therapy; abdominal therapy; and pelvic therapy.

AI or machine learning technologies can be used for a variety of functions of the invention according to various embodiments. For example, operation of the imaging, diagnostic, and therapy delivery functions as discussed above, but also rapid disease diagnosis based on the information obtained by the remote diagnostic devices and sensors. AI and/or machine learning technologies can also be used to assist a remote operator instead of providing direct control (e.g., by suggesting areas to image or additional diagnostic tests to be performed). AI and/or machine learning technologies and software engines can also be applied to test ordering, appointment scheduling (e.g., to maximize efficiency or pair patients with remote operators based on time, test proficiency, costs, language, or compatibility), test and/or therapy triage when device availability is limited, and device transportation. Exemplary machine learning or AI techniques or algorithms contemplated herein can include supervised or unsupervised learning algorithms and may include naïve bayes, support vector machine (SVM), random forest, linear regression, logistic regression, k-nearest neighbor (KNN), k-means, decision tree, classification and regression tree (CART), Apriori, principle component analysis (PCA), CatBoost, iterative dichotomiser 3(ID3 ), hierarchical clustering, back-propogation, adaptive boosting (AdaBoost), deep learning, gradient boosting, Hopfield network, and C4.5.

Upon completion of the medical procedure (e.g., gathering of diagnostic information and/or provision of treatment), the patient may be instructed to return the device. In certain embodiments, portions of the medical device including, for example, sensors such as an ultrasound patch, may be disposed of. In preferred embodiments, portions of the sensors or medical device that directly contact the patient may be disposable to avoid biohazards and/or the spread of communicable diseases. Some portions of the hardware of the medical device and/or the transmitter or console may be returned via the same services as used for initial delivery to the patient (e.g., via traditional couriers, EMS, drones, or third-party delivery apps or services). In certain embodiments, any delivered medical devices or other equipment may include software or mechanical features that lockout subsequent use after completion of the authorized medical procedure (e.g., testing or delivery of therapy). These security features may be automatic to limit the use of the devices or may be initiated remotely through the remote workstation. Such lockouts can require reset by the device owner or service provider to discourage a patient or other individual from stealing or otherwise misusing any of delivered device. In certain embodiments, the delivered devices or components thereof may self-destruct or “brick” themselves upon tampering. For example, a therapeutic medical device may contain one or more drugs for authorized delivery to a patient. Such drugs may be dangerous if used outside of the authorized treatment regime. Accordingly, should the drug compartment of the medical device be tampered with, the device may release a solvent, a chemical, a concentrated pyrotechnic, or other destructive device to destroy or render the excess drugs in the compartment unusable or inert. In certain embodiments, security features may include a tracking device such as a GPS tracking device so that the location of any delivered equipment can be remotely monitored and shipments can be tracked.

While systems and methods of the invention are exemplified herein by remote ultrasound imaging, one of ordinary skill in the art will recognize that any number of additional diagnostic devices and procedures can be incorporated into the described systems and methods and are contemplated herein. For example, automatic or remotely operated diagnostic sensors/tests may include impedance; plethysmography; optical coherence; infrared mapping; acoustic signature/profiling (in addition to imaging also detects abnormal sounds including auscultation of heart and other organs); EKG—electrocardiography; EMG—electromyography; EEG—electroencephalography; physiologic monitoring such as pulse, BP, ECG, and oxygen saturation; elastography—tests skin and tissue elasticity and other mechanical properties (e.g., as performed in breast to detect potentially cancerous tissue—or in bone); raman spectroscopy; or mechanically scraping of skin or other tissue surface to “biopsy” that area. In certain embodiments, collected tissues can be automatically stored within the device under controlled conditions (e.g., secured, temperature controlled, and sterile containers) for subsequent analysis upon return of the device.

In various embodiments, the medical device and/or sensor to be delivered to a patient and/or remotely operated may comprise an ultrasound sensor as described in detail below and in U.S. Pat. No. 8,038,622, incorporated herein by reference. While systems and methods of the invention are exemplified herein by remote ultrasound imaging, one of ordinary skill in the art will recognize that any number of additional diagnostic devices and procedures can be incorporated into the described systems and methods and are contemplated herein. For example, automatic or remotely operated diagnostic sensors/tests may include impedance; plethysmography; optical coherence; infrared mapping; acoustic signature/profiling (in addition to imaging also detects abnormal sounds including auscultation of heart and other organs); EKG—electrocardiography; EMG—electromyography; EEG—electroencephalography; physiologic monitoring such as pulse, BP, ECG, and oxygen saturation; elastography—tests skin and tissue elasticity and other mechanical properties (e.g., as performed in breast to detect potentially cancerous tissue—or in bone); raman spectroscopy; or mechanically scraping of skin or other tissue surface to “biopsy” that area. In certain embodiments, collected tissues can be automatically stored within the device under controlled conditions (e.g., secured, temperature controlled, and sterile containers) for subsequent analysis upon return of the device.

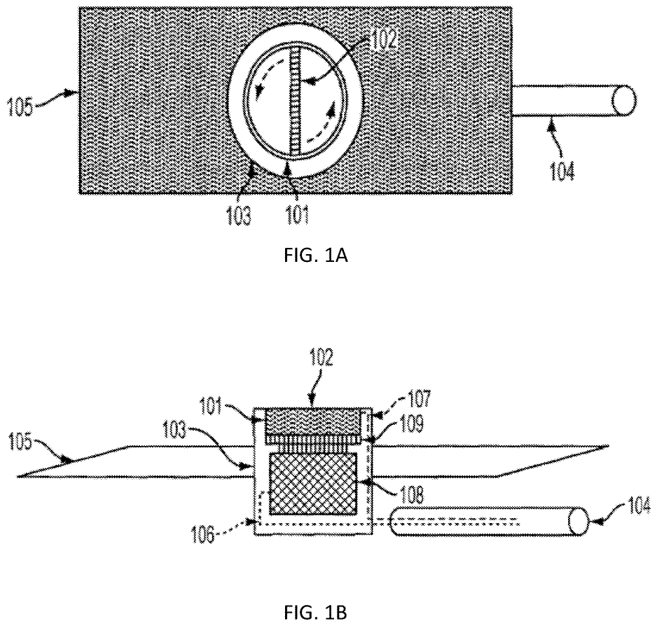

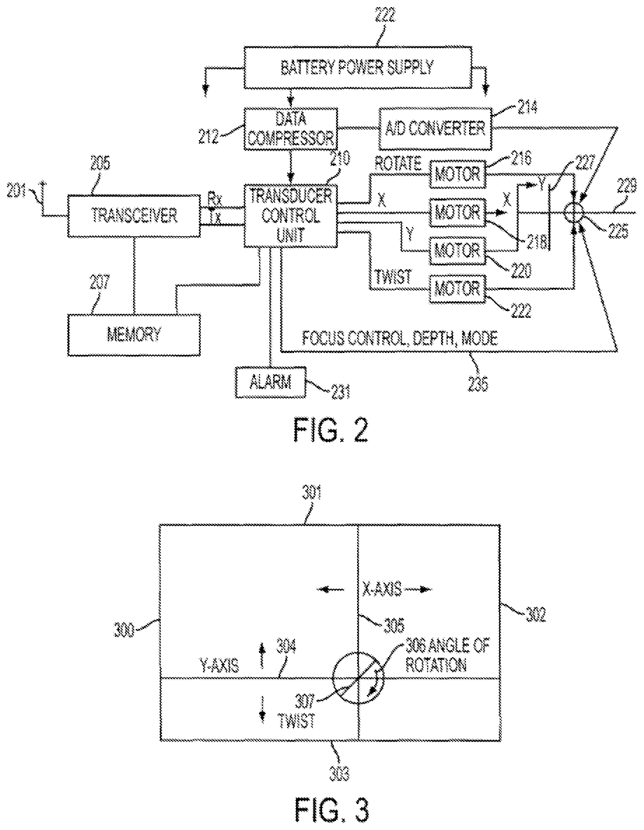

FIG. 1 comprises a top view and side view of a first plurality of embodiments and aspects of a multi-plane transducer unit comprising a rotatable linear array of transducer elements including a housing. The housing may be mounted by securing material to a body of, for example, a patient or victim. The transducer array or element may be remotely controllably rotated and otherwise remotely controlled by wired or wireless signals transmitted toward the array from a remote workstation. An operator need not be proximate the patient's body to manipulate or control the transducer elements or housing. Similar reference numbers will be used throughout the detailed description to refer to similar elements wherein the first number of the reference number denotes the figure in which an element first appears. Transducer 101 may comprise a single transducer element for ultrasound transmission and reception of reflected sound waves or a linear array 102 of transducer elements mounted, for example, in a circular manner from a top perspective as a diameter of the circle or at the center of the circle comprising housing 103. An arrow indicates an angle of rotation in a counter-clockwise direction of the transducer element or a linear array 102 within housing 103. Typically, an angle of rotation of 180 degrees when used with a linear array 102 will permit the collection of a plurality of image planes, for example, of the heart over which the array within housing 103 may be located and fixed to the body surface, in this case, a cylindrical housing as seen from top and side views forming a circular footprint on the body surface. The housing 103 is fixed to the surface of a human body, for example, in a position at the center of the chest to monitor the heart immediately below. The top surface of the side view shows transducer 101 which may rotate within the housing 103. The top surface of housing 103 intended to be fixed to a patient contains an impedance matching substance which may be complimentary to the application of a suitable impedance matching gel. Fastening or securing material 105 is shown in top and side views for fixing the housing to a human body skin surface with the transducer/impedance matching surface facing the human body surface. Within the housing 103 is also contained at least one motor, in the embodiment of FIG. 1, a motor 108 for rotating a transducer element or linear array 102. Also located within the housing 103, for example, in the vicinity of the motor may be a wireless transceiver and antenna (not shown; see, for example, FIG. 2) and other circuitry as necessary for receiving motor control signals and other known control signals such as on/off, mode, depth, focus and the like. Also, not shown is a battery or power system for powering the motors and circuits requiring power. Alternatively to a wireless device, housing 103 may have a control cable or wire 104 for transducer output, power, motor control and the like.

Cable 104 may lead to a workstation console, preferably remote from a patient bedside and operate in a similar manner to known cables used with devices such as Toshiba Power Vision™ ultrasound machine, the difference being that the depicted cable further includes a rotation motor control lead or leads or a data line of such cable further incorporates motor control data in a serial data stream. Cable 104 may include motor wiring 106 to rotation motor 108 for control and power purposes. Cable 104 may further comprise transducer wiring 107 for power, control and image collection purposes.

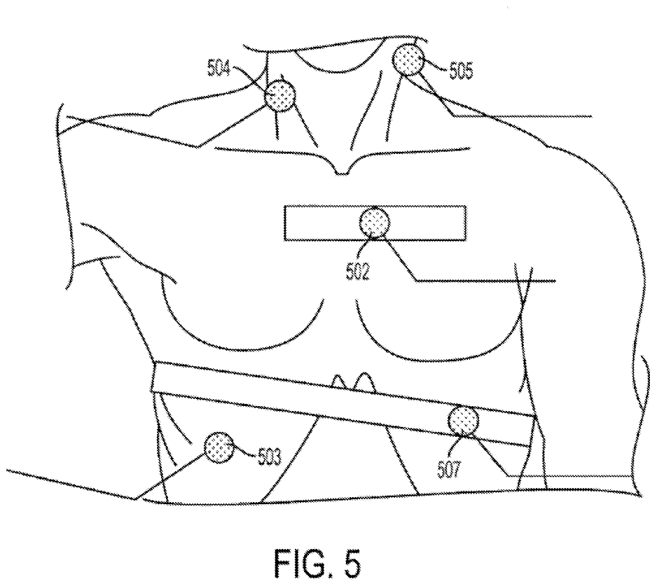

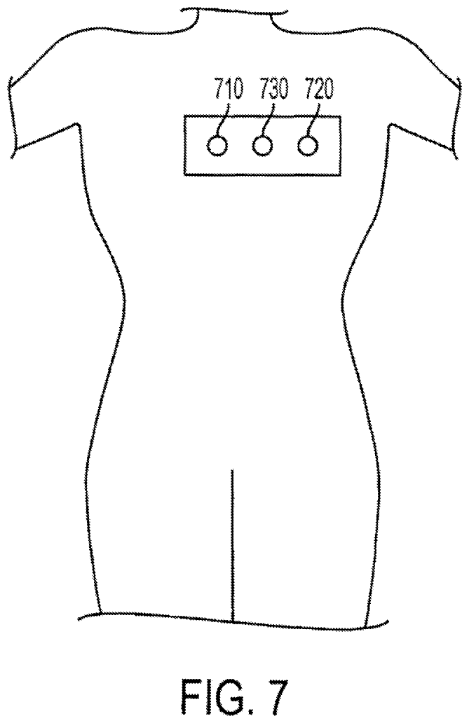

As will be described herein, further motors 108 may be provided for twisting linear array 102 to permit a different direction of sound wave emission and/or reception, and for providing two directions, for example, lengthwise and widthwise (x and y) axis movement in the plane of the human body surface and according to how a rectangularly shaped housing 103 is placed on the body, i.e. an x and y axis are considered in relation to the housing. The housing 103 may be mounted at an angle (see, for example, manner of fixation 501 or 502 of FIG. 5 to the human body). Motor 108 may comprise an optional gear assembly 109 for more accurate, for example, incremental movement of array 102. Motor 108 is preferably a micro or miniature linear motor known in the art for turning a rotor and optional gear assembly for rotating the coupled transducer element or linear array 102 at incremental steps such as one degree steps from a vertical or horizontal orientation (vertical shown) through 180 degrees—clockwise or counterclockwise. In this manner, a linear array 102 may capture 180 different planes of view of, for example, a heart under observation, and a three dimensional view may be constructed using known software data analysis processes. Of course, the three dimensional analysis is improved and made stereoscopic if pairs (or more than two arrays) of devices at different observation locations according to FIG. 1 are used as will be described in conjunction with a discussion of FIG. 7. A transceiver (not shown) or a cable 104 may report the actual position of the linear array to a remote workstation (not shown) as a value, for example, between 0 and 180 degrees.

Typical sizes for a cylindrical transducer housing 103 as shown in FIG. 1 may be from 1 cm in diameter to 3 cm in diameter. The height of the cylindrical housing may be similar or less than 1.5 cm.

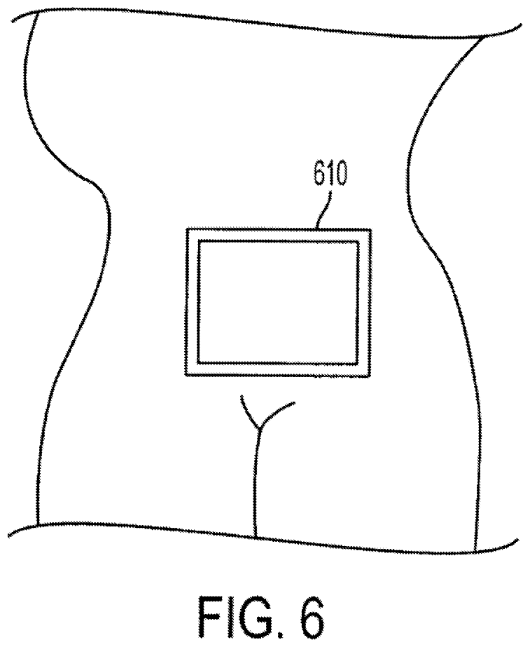

If a housing has a rectangular shape, for example, for observation of a fetus (see FIG. 6), its shape may be one the order of size of 10 cm by 12 cm, in which case, linear motors are provided for two directions, for example, x and y axis manipulation of the transducer array in addition to rotation. Referring briefly to FIG. 3, the surface proximate to the body of a housing 103 may comprise a rectangular shape and linear motors may move small rods carrying, for example, a transducer array to a particular x, y coordinate ranging from 0 to 10 cm in one direction to 0 to 12 cm in the other direction within its footprint on the body surface in incremental steps, for example of 5 mm. In a further embodiment as described above, a motor may be provided and mounted to twist a linear array as well as provide an incremental angle of rotation, again, within a range of 0 to 180 degrees with a default position at 90 degrees, or directly pointing sound waves into the human body.

FIG. 2 provides a schematic block diagram for embodiments and aspects of a wireless device as shown in FIG. 1 including a transceiver (which may be a wireless telecommunications transceiver), a transducer control unit, a battery, at least one motor for rotating a linear transducer element array, the linear transducer element array and analog to digital circuitry for converting collected image data to digital form for transmission via the transceiver. In FIG. 2, a wireless embodiment of a remotely manipulatable ultrasound transducer is assumed. Battery supply unit 222 is preferably a rechargeable lithium battery known in the art that powers all units requiring power within a housing 103.

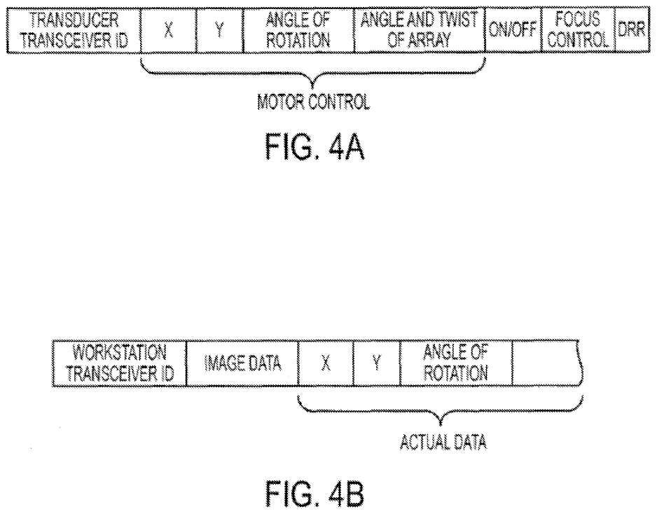

Transceiver 205 is an alternative to a control cable 104 for transmitting and receiving information and may receive and transmit a digital data signal generally in keeping with FIGS. 4a and 4b via antenna 201. While these figures depict what may be construed as a serial data stream, the depicted data may be sent in parallel or serial format and in any order including the order shown. Known telecommunications protocols may be utilized if the transceiver transmits and receives by radio frequency signal such as WiFi, blue tooth, Wimax and the like for a wireless local area network. As is known in the art, infrared and ultrasound may be used as well as other light frequencies than infrared. On the other hand, light waves are typically incapable of penetrating through walls and require a line of site transmission path. Yet, by way of example, light wave transmission is feasible; for example, a light wave transceiver connected to a workstation may be mounted, for example, in the ceiling of an operating arena and a unit mounted to a patient facing upward may communicate with the ceiling mounted unit in a line of site. As described above, since a cable 104 provides a direct link to a remote workstation, cable 104 need not necessarily transmit data uniquely indicative of a given transmitter, transducer or workstation because the cable 104 may comprise a direct link between known devices. If any other device is connected to cable 104, then addressing using a unique address (or telephone number) or other identifier should be used for a connected device. Transceiver 205 may receive a data signal from a workstation, demodulate the signal and output a demodulated baseband data signal including data per FIG. 4a to controller 210 which may be a microprocessor, application specific integrated circuit or other control circuit which may be designed and fabricated in a manner well known in the art. In the other direction of transmission, the transceiver 205 may receive image data for one or more planes or sequential images and other signal including actual position data per FIG. 4b from controller 210 for transmission to a uniquely identified remote workstation.

Following the path of a received signal at antenna 205, the received signal may be received at radio frequency at transceiver 205, demodulated and a Rx data output signal passed to controller 210 for processing. Controller 210 authenticates the signal as directed to it by means of the transmitted unique transducer identification code of FIG. 4a. In addition, the signal may require processing in accordance with well known protocols for decompression, decryption, parity and other data error detection and correction algorithms and the like (not shown). In one embodiment, for example, for multi-planar imaging purposes, the transducer array 225 is linear and may be rotated. A rotate signal which may indicate an angle between 0 and 180 degrees in incremental steps of, for example, one to five degrees can indicate rotation in a clockwise or counterclockwise direction or indicate an angle to which the transducer array or element is to be rotated (for example, from 90 degrees, actual present position, to 120 degrees, desired position) is received and passed to linear motor 216 having a rotor for rotation using, possibly, an optional gear assembly 109 for turning the linear array 102 to a desired angle of rotation.

In an alternative embodiment, for example, for therapeutic purposes, a direction of sound wave propagation, depth and the like signal are received and reported to actuate twist motor 222 to a desired angle of twist in addition to a desired angle of rotation via motor 216 to, for example, deliver a therapeutic sound wave to a given body organ or sub-tissue layer at a given transmitted depth, for example, represented by a sound wave power level, within the patient's body from the transducer 102, 225. In an embodiment paired with another unit, the angle of twist and rotation may be synchronized so that one transducer array 102 may cooperate with another transducer array as sound wave transmitter and sound wave receiver for together providing image data either individually or together.

In a further alternative embodiment, the transducer array 102 or transducer element may be manipulated in two directions, perpendicular to one another, along the patient's body surface, denoted an x direction and a perpendicular y direction or axis as shown in FIG. 3. The transceiver 205 outputs such control data to controller 210 which then actuates motors 218 for x axis movement and 220 for y axis movement of transducer element or transducer array 102, 225 as shown in FIG. 3. Also shown in FIG. 2 are x, y axis 227, 229 which are controlled by motors 218, 220. When arriving at the x, y position of interest, the transducer 102, 225 may be rotated or twisted or rotation and/or twisting/rotation may occur en route to the x, y position of interest. Feedback to the remote workstation may be provided via actual data indicating all parameter values of interest, on/off, focus level, depth, x axis, y axis, angle of rotation and angle of twist (most of which are shown in FIG. 4b).

Also, controller 210 may be in receipt of off/on, focus control, mode, depth and other control data which is passed to transducer 102, 225 for proper operation, for example, to regulate the amount of power delivered to transducers for sound wave emission or for focusing the array. This control lead or collection of leads is shown as data line 235.

The output of transducer array 102, 225 may be raw image (reflected sound wave) data similar to that obtained by a hand-held transducer array known in the art. It may be in analog form and provided to an A/D converter 214 for sampling at an appropriate sampling level. The data signal output of A/D converter 214 may be further compressed at data compressor 212 prior to formatting at controller 210 for transmission at transceiver 205 and/or storage at memory 207. These circuits 214 and 212 are shown as separate circuits but may, together with controller 210 be in the form of a single application specific integrated circuit (ASIC) or provided as separate circuits. Memory 207 may be on board a microprocessor chip or provided separately. In one embodiment, memory 207 may comprise a removable memory for uploading data to a device for telecommunications transmission. The image and other data prior to transmission or for long term storage may be temporarily or more permanently stored in memory 207. Similarly, memory 207 may be utilized for temporarily storing control data as received from transceiver 205 and prior to being operated on by controller 210. In one embodiment as will be described herein, there is no data transmission via cable or wireless means.

Image and associated position data and the like for a given image along with time of day and date may be stored in a fanny pack or personal remote control device worn or otherwise carried by the patient. This assumes a time of day and date clock associated with controller 210 or the time and day may be periodically updated via a transmission to the unit of FIG. 2. In, for example, a therapeutic embodiment of a remotely manipulatable transducer array, the patient wearing or carrying the device may control delivery of therapeutic sound waves via a transducer array 102 and control the direction and depth of transmission. For example, ultrasound has been found to assist in relieving arthritis and other pain, for example, in a hip, shoulder, knee or other joint.

In one embodiment where the circuitry and motors are contained in a housing and in accordance with FIGS. 1, 2, 3 and 4, the housing is worn by a person, the person may be remotely observed as they go about their daily routine. For example, a remotely manipulatable ultrasound transducer array located so as to monitor a major organ may detect a change that requires medical attention. In such an instance, alarm 231 may comprise a vibrator or display or other alarm device to signal the wearer to report to a facility. The alarm may also indicate a point in time when a memory 207 is full of un-transmitted images, and the wearer must change their memory card of memory 207 or report to a workstation or other telecommunications facility for image data upload.

FIG. 3 provides an overview of a mechanical arrangement to be contained within a housing 103 of rectangular or square embodiments of a transducer unit for manipulating a transducer or linear transducer array in two directions, for example, along an x axis and a y axis and to provide an angle of rotation and a twist angle at a desired x, y coordinate pair to redirect a sound wave emitted by a transducer or linear array of transducer elements whereby it is envisioned that a footprint on a patient body surface is rectangular or square and relates to the embodiments and circuits of FIGS. 1 and 2. Assume the rectangle housing comprises guide wires or rods 300, 301, 302 and 303 on which are provided y-axis rod 304 which may be moved in an up and down direction shown via a corresponding motor 220 and gear assembly not shown to incremental steps along the y axis. Similarly, there is provided x-axis rod 305 which may be moved to the left or the right direction shown via corresponding motor 218 and a gear assembly not shown. X-axis rod 305 and Y-axis rod intersect at a desired point where an array or element may be affixed via further motors 216, 222. For example, rotor 306 of motor 216 (in combination with an optional gear assembly 109) provides rotation of a mounted transducer array 102, 225 or transducer element to a predetermined or desired angle of rotation. Motor 222 provides twist 307 to linear array or element 102 to change direction of sound wave transmission or reception with 90 degrees-straight down-being a default position for twist.

FIG. 4a provides an exemplary signal content format for providing motor control of a transducer or transducer array of FIG. 1, 2 or 3 in a direction from a workstation to a remote wired or wireless transducer including a unique transducer transceiver identifier if wireless transmission is utilized. The format also provides for known control such as on/off, focus, depth, mode and the like. Other control data may come to mind of one of ordinary skill in the art of ultrasound apparatus. Motor control data may be transmitted, for example, in the form of ultimate desired position or as an incremental step from an actual position or other way that may come to mind of one of skill in the art.

FIG. 4b provides an exemplary signal content format for providing a reply signal from a transducer or transducer array of FIG. 1, 2 or 3 in a direction from a remote wired or wireless transducer including a unique workstation identifier if wireless transmission is utilized. The format provides for feedback of actual location data of the position of the transducer or transducer array. The actual location data may be compared to a desired location to determine if the remotely manipulatable transducer or transducer array has reached a desired position so that imaging may begin.

FIG. 5 provides a plurality of suggested locations on a human body for locating a remotely manipulatable ultrasound transducer or transducer array whereby the units may comprise housings and utilize adhesive material similar to materials used for applying electrodes to a human body as used in electrocardiography. Also, banded adhesive attachments to housings or bandage or banding of the manipulatable transducer or transducer array housing to the human body may be provided. No position on the human body should be considered to be excluded from reach by a remotely manipulatable transducer or transducer array. Fixing material for a given position on the patient's body is well known in the field of bandaging from first responder training in medical emergencies and the like.

Image locations 507 and 502 may be used in combination to provide image locations for the heart and develop three dimensional images thereof in multiple planes. Location 503 may be used to image the liver. Image location 504 may be used to image the carotid artery or neck structure or any gland within the neck. Image location 505 may be used to image trans-cranial structures including blood vessels. The fixing material may be a band as in 507 or adhesive tape material as in 502 or affixed via an adhesive as in 503, 504 or 505.

Now some specific applications of the remotely manipulatable ultrasound transducer apparatus of FIGS. 1-5 will now be discussed with reference to FIGS. 6 and 7. FIG. 6 represents a fetus imaging and FIG. 7 an imaging during a heart operation.

### Fetus Monitor

FIG. 6 provides an exemplary application of a manipulatable transducer array that may be worn by a pregnant female in the abdominal area for remotely monitoring a fetus for, for example, extraordinary conditions. Let us assume that a female patient has been checked into a maternity ward of a hospital. A rectangular, remotely manipulatable transducer array may be affixed as shown in FIG. 6 by adhesive to the abdominal cavity and appropriately be provided with a surface that is sufficiently curved as to maintain contact with a, for example, 10 cm by 12 centimeter rectangular region of interest. For example, such surface of the housing 103 may be made of a giving material such as rubber and the transducer assembly contained within the housing at sufficient depth to permit the curved skin surface to be absorbed within a curved surface and maintain constant skin contact. The patient typically is prone to considerable movement during pre-birth and requires considerable attention. The housing must be firmly secured by adhesive material. Fetus monitoring equipment may be used in concert with the transducer array and the fetus remotely monitored for activity such as turning or stage of birth canal entry and passage via the remotely manipulatable transducer or transducer array shown. Typically, bed-side space is at a premium as the father or other coach and nurses are present at bedside. Also, periodically, a physician or surgeon will want to check the degree of opening of the birth canal for delivery. A rectangular housing 610 affixed as shown in FIG. 6 permits remote monitoring of a fetus during pre-birth and birth from a remote workstation, saving valuable bed-side space and providing an invaluable supplement to normal fetus monitoring devices, for example, for monitoring fetus heart rate and pressure.

### Minimally-Invasive Pericardial Surgery

FIG. 7 shows an arrangement for pericardial activity and possibly surgery including first and second remotely manipulatable transducer units, 710, 730 mounted to either side of, for example, an image guided catheter 720 surgical location as one example of an application for minimally invasive heart surgery. The image guided catheter may be one as shown and described in my co-pending U.S. patent application Ser. No. 11/782,991, filed Jul. 25, 2007, entitled “Image Guided Catheters and Methods of Use,” of the same inventor, for example, per FIG. 1 of that application, incorporated herein by reference as to its entire contents. Methods of use include, for example, double balloon heart wall processes, cavity drainage and the like wherein the wireless arrays 710, 730 may image successful deployment of the image guided catheter and intervention, therapeutic or diagnostic apparatus in the region of interest of the heart. Elements or transducer arrays 710, 730 may cooperate to provide stereoscopic as well as multi-planar imaging while the image-guided catheter contains its own ultrasound array for imaging a point of intervention, therapeutic or diagnostic care.

A workstation for use with such an arrangement shown in FIG. 7 may comprise a plurality of screens to show all images obtained from each of ultrasound transducer elements or arrays 710, 720 and 730. An operator remote from the operating suite may remotely manipulate and control arrays 710, 730 and obtain imaging therefrom. The workstation operator may communicate with a surgeon by a wireless telecommunications device worn, for example, as a headset by each of the surgeon (or other operating room personnel) and the workstation operator. The transducers may be manipulated remotely within a region of interest remotely from the workstation and remain on the patient during the entire operation. The imaging can occur when and as requested by the surgeon of the workstation operator. Uses may comprise, for example, but not be limited to pericardiocentesis, vascular surgery, coronary angioplasty, valvuloplasty, alcohol septal ablation, delivery of drugs, stem cells or laser therapy, valve repair or replacement, cardiac shape modifying devices such as ACORN-like or MYOSPLINT™, myocardial scar reconstruction, ventricular reconstruction and ventricular assist device placement. One may monitor the blood flow of any vessel to and from the heart or carotid blood flow during cranial or other surgery. Besides the image guided catheter, other devices usable with a remotely manipulatable transducer or transducer array include a biopsy forceps, a drainage catheter, a pressure monitoring system, a suture application system, a therapy delivery catheter or system or other intervention system.

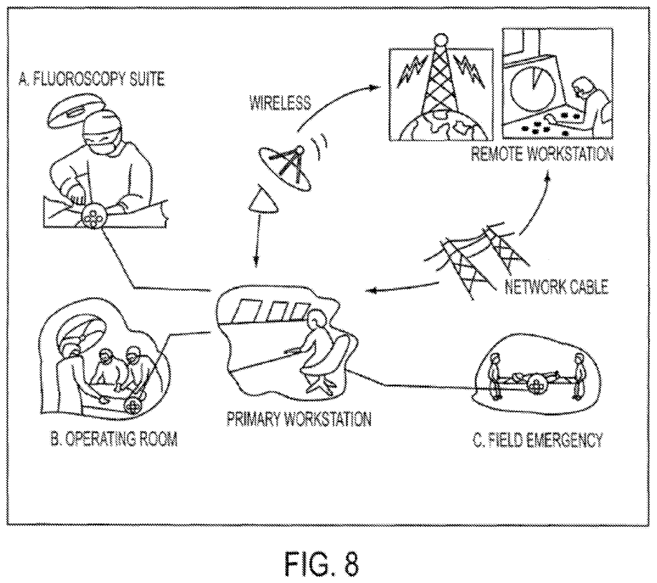

FIG. 8 shows a workflow scenario whereby the primary workstation is remote from a fluoroscopy, operating room and the like and, if necessary, shielded from adverse impacts of radiation such as magnetic resonance. One further limitation of using ultrasound in an operating suite is electrocautery procedures which may degrade collected image signal quality when the electrocautery apparatus is in use. Referring to FIG. 8, there may be situations where the ultrasound operator be optimally protected, for example, in a fluoroscopy suite where the operator is exposed to radiation and would otherwise need a heavy lead suit. Even with the lead suit, the operator would typically have to move a 400 or 500 pound ultrasound machine back and forth from along side the operating table to away from the table when a C arm is being used. Now, the ultrasound operator may sit at a remote workstation after placing the remotely manipulatable transducer or transducer array on the surgical patient and then sit behind a lead shield to manipulate and operate the transducer or linear transducer array remotely at their primary workstation. Also, there is typically inadequate space in an operating room for an ultrasound operator, for example, next to a surgical operating table. The operator may place the transducer and then remotely manipulate and control and view images from the remotely manipulatable transducer. Their workstation can be located in a corner of the operating room or outside the operating room and the operator communicate by telecommunications means with the surgeon (or other operating room personnel).

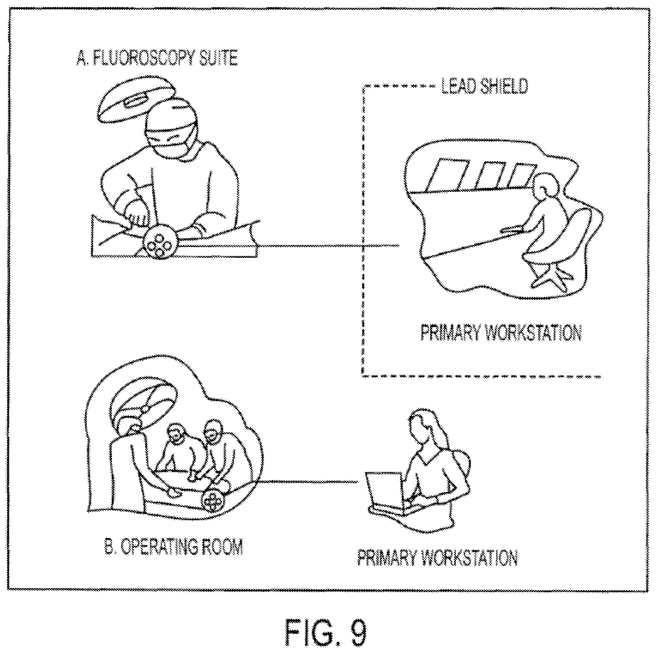

FIG. 9 shows another workflow scenario for describing, for example, remote field emergency use, operating room, fluoroscopy suite use and primary and alternative workstations connected by a wireless or wired network such as a telecommunications network. Regardless of the patient location, even if the patient is considered a field emergency, the remotely manipulatable transducer may be placed by an emergency first responder, remotely manipulated to a desired location on the patient body surface and image data remotely transmitted to a remote workstation. The emergency first responder can respond to simple commands to appropriately place one or more transducers on the victim, for example, via a telecommunications channel. For example, first responder personnel at a remote location in Alaska where there is a medical emergency can be remotely guided by a hospital in Juneau to properly place a remotely manipulatable transducer or transducer array at a location on the body surface, and the image data may be conveyed by satellite telecommunications to Juneau as control data is transmitted on the reverse path from a remote operator/manipulator. A second communications channel may used by the operator to guide the first responder as to the proper placement of the remotely manipulatable transducer or transducer array. Thereafter, the remote operator in Juneau may move the array remotely and control the diagnostic, therapeutic or interventional imaging.

What is claimed is:

1. A method for performing remote medical procedures, the method comprising:
providing a medical device comprising a sensor in communication with a transmitter comprising a processor and a tangible, non-transient memory connected to a network, the medical device operable to obtain diagnostic tests on a patient;
initiating communication between the transmitter and a remote workstation;
receiving patient information from the sensor at the remote workstation;
providing commands to the medical device from the remote workstation to control the medical device to perform a medical procedure.

2. The method of claim 1, further comprising instructing the patient to setup the medical device via the transmitter.

3. The method of claim 1, wherein the transmitter is a patient computer, the method further comprising providing software to the patient computer to communicate with the remote workstation and medical device.

4. The method of claim 1, further comprising delivering the medical device to the patient.

5. The method of claim 4, wherein the medical device is delivered by a drone.

6. The method of claim 4, wherein the medical device is delivered by a self-driving vehicle.

7. The method of claim 1, wherein the medical device comprises a security feature.

8. The method of claim 7, wherein the security feature comprises a lockout preventing subsequent use of the medical device after completion of the medical procedure.

9. The method of claim 7, wherein the security feature comprises a self-destruct mechanism rendering the medical device inoperable upon unauthorized disassembly or use of the medical device.

10. The method of claim 7, wherein the security feature comprises an electronic tracking mechanism.

11. The method of claim 1, wherein the sensor is selected from the group consisting of an ultrasound device, an impedance sensor, a plethysmography device, an optical coherence device, an infrared sensor, an acoustic sensor, an electrocardiogram electromyography device, an electroencephalography device, a pulse sensor, a blood pressure sensor, an oxygen saturation sensor, an elastography device, a Raman spectrometer, and a tissue collecting device.

12. The method of claim 11, wherein the sensor comprises a tissue collecting device, the medical device further comprising a sterile and temperature-controlled tissue storage container, the medical device operable to store a collected tissue sample from the tissue collecting device in the storage container.

13. The method of claim, wherein the medical device is further operable to deliver a therapy to the patient, the method further comprising providing commands to the medical device from the remote workstation to deliver the therapy to the patient based on the patient information received from the sensor.

14. The method of claim 13, wherein the therapy is selected from the group consisting of delivery of thermal energy to the patient; oral, transdermal, intravenous, or inhalation delivery of a therapeutic compound to the patient; iontophoresis-aided drug delivery; radiation therapy; chemotherapy; clotting therapy; anti-clotting therapy; laser ablation; phototherapy; acupuncture; electrical stimulation; magnetic stimulation; topical therapy; oral therapy; dental therapy; eye treatment; musculoskeletal therapy; abdominal therapy; and pelvic therapy.

15. The method of claim 1, wherein one or more of the receiving patient information and providing commands steps are performed by a technician at the remote workstation.

16. The method of claim 1, wherein one or more of the receiving patient information and providing commands steps are performed automatically by a computer comprising a non-transitory memory and a processor.

17. The method of claim 16, wherein the computer provides the commands based on an analysis of the patient information received using one or more artificial intelligence algorithms.
